(19) United States
(12) Patent Application Publication
(10) Pub. No.: US 2008/0262443 A1
(43) Pub. Date: Oct. 23, 2008

(54) AUTOMATIC INJECTOR SEQUENCE CONTROL FOR A CONTAINER REPLACEMENT

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/113,587

(22) Filed: May 1, 2008

Related U.S. Application Data
(63) Continuation of application No. PCT/CH2006/000604, filed on Oct. 31, 2006.

(30) Foreign Application Priority Data
Nov. 3, 2005 (DE) 10 2005 052 460.5

(51) Int. Cl. A61M 5/00

(52) U.S. Cl. 604/264

(57) ABSTRACT

An injection device, e.g. an automatic injector, including a mechanism holder, an advancing element that can be displaced along a longitudinal axis in relation to the mechanism holder, an elasticity element for driving the advancing element, at least one blocking element that is coupled to the advancing element and can engage in the mechanism holder to prevent a longitudinal movement of the advancing element in relation to the mechanism holder, and a locking element that can be displaced along the longitudinal axis in relation to the advancing element and, when in a blocking position in front of the at least one blocking element, blocks the at least one blocking element such that it does not disengage from the mechanism holder. A method of performing or controlling an injection sequence is encompassed.

### CROSS-REFERENCE TO RELATED APPLICATIONS

This application is a continuation of International Patent Application No. PCT/CH2006/000604 filed Oct. 31, 2006, and claims priority to German Application No. DE 10 2005 052 460.5 filed Nov. 3, 2005, the contents of both of which are incorporated in their entirety herein by reference.

### BACKGROUND

The present invention relates to devices for delivering, dispensing, injecting, administering or infusing a substance, and to methods of making and using such devices. In some embodiments, the present invention relates to an injection device for administering an injectable product, such as a liquid drug such as insulin, heparin, a growth hormone or an osteoporosis preparation. The injection device may take the form of an injection pen.

German utility model specification 203 19 648 discloses an injection device which comprises a mechanical sequence controller. The injection process comprises an injection sequence and a delivery sequence. During the injection sequence, an advancing structure is moved in an injecting direction, such that a needle of the device is injected into a body tissue. The delivery sequence is started once the injection sequence has been completed, wherein a piston rod is advanced in the delivery direction and drives a piston in an ampoule, which delivers the product through the injected needle into the body tissue.

### SUMMARY

It is an object of the present invention to provide an injection device in which the injection sequence can be performed more reliably and the device can be easily prepared for a new injection. It is another object of the present invention to provide a method for reliably preparing an injection device for a new injection. It is another object of the present invention to provide a method by which such a device can be made and/or operated.

In some embodiments, the present invention comprises an injection device including a mechanism holder, an advancing element that can be displaced along a longitudinal axis in relation to the mechanism holder, an elasticity element for driving the advancing element, at least one blocking element that is coupled to the advancing element and can engage in the mechanism holder to prevent a longitudinal movement of the advancing element in relation to the mechanism holder, and a locking element that can be displaced along the longitudinal axis in relation to the advancing element and, when in a blocking position in front of the at least one blocking element, blocks the at least one blocking element such that it does not disengage from the mechanism holder. A method of performing or controlling an injection sequence is encompassed.

In some embodiments, an injection device in accordance with the present invention is an automatic injection device, which also might be known as or referred to as an auto-injector. In such embodiments, the administering of a drug can comprise an injection sequence in which a needle is injected, or driven or inserted, into a body tissue and a delivery sequence in which the drug is delivered. Using the present invention, it is possible to securely separate the delivery sequence and the injection sequence from each other, such that the delivery sequence can only be performed once the injection sequence has been performed. In some embodiments, the device can comprise a housing which comprises two housing parts which can be detachably or non-detachably connected to each other. A screw lock, bayonet lock, latching lock or other suitable connector may be considered for this purpose. A mechanical sequence controller can be accommodated in a proximal or rearward housing part, and a product container can be accommodated in a distal or forward housing part. “Proximal” is understood to mean the side or end opposite the needle, and “distal” is understood to mean the side or end of the injection device, which typically is elongated, which lies at the needle. The product container, e.g. an ampoule, can be inserted, such that it can be exchanged, into the distal housing part or can be formed directly by the distal housing part.

In some embodiments, the injection device, e.g. the proximal housing part, comprises a mechanism holder which can be connected to the housing, fixedly or such that it cannot be shifted. The mechanism holder can be inserted, as a separate part, into the housing and connected, axially and rotationally fixed, to the housing. Alternatively, the mechanism holder can be integrated with the housing. In some embodiments, the mechanism holder is sleeve-shaped and can mount or carry an advancing element which can be moved along a longitudinal axis of the injection device relative to the mechanism holder.

In some embodiments, the advancing element can be sleeve-shaped and its outer circumferential surface can slide along an inner circumferential surface of the mechanism holder. In some preferred embodiments, the advancing element can be driven by an elasticity element, e.g. a helical spring, for a movement relative to the mechanism holder, when the advancing element is released for moving relative to the mechanism holder. The elasticity element may be supported on the mechanism holder and on the advancing element, wherein the spring, which is or can be pressurised, generates the drive force for the advancing element. The spring can surround the advancing element over a certain length, and/or be at least partially accommodated in an annular gap formed by the advancing element and the mechanism holder. The advancing element can comprise an annular flange at its distal end, on which the elasticity element can be supported.

In some embodiments, the advancing element can be blocked or prevented from moving relative to the mechanism holder by at least one blocking or lock element which is coupled to the advancing element, when the blocking element engages with the mechanism holder. The blocking element can be coupled to the advancing element such that it can only perform a movement relative to the mechanism holder in an approximately radial direction in relation to the longitudinal axis and substantially cannot perform movements in the axial direction. In some embodiments, the blocking element can be a sphere which can be radially moved back and forth in a radially extending breach or receiving structure in the advancing element. The blocking element may be cam-shaped or tapered and elastically connected to the advancing element via an arm. The blocking element, the arm and the advancing element may be integrated. The mechanism holder can comprise a blocking groove for each of the at least one blocking elements, with which the at least one blocking element can engage. The mechanism holder comprises an annular blocking groove with which the at least one blocking element can engage.

In some embodiments, the blocking element can be formed such that it can be moved counter to the injecting direction of the needle, out of the engagement with the blocking groove, when the advancing element is moved or a pressure is exerted on the advancing element. To this end, the blocking element can comprise a surface which can slide off on a flank of the blocking groove and move the blocking element out of the engagement. The blocking element can also be moved out of the engagement with the blocking groove by the force of a spring, e.g. by elastically arranging the blocking element on an arm.

In some embodiments, a latching element is also provided which can be moved along the longitudinal axis of the injection device, relative to the advancing element, wherein in a position in front of the at least one blocking element, the latching element blocks or prevents a movement of the blocking element out of the engagement with the mechanism holder. When the latching element crosses the direction of movement with which the blocking element would be moved out of the blocking groove, the engagement between the blocking element and the mechanism holder is secured, irrespective of a magnitude of the force applied to the advancing element. In this position, the blocking element is situated between a latching portion of the latching element and the groove base of the blocking groove. The latching element can be mounted or carried, such that it can be shifted, by the advancing element. The advancing element can comprise at least one groove, in some preferred embodiments, a number of grooves, which extend along the longitudinal axis and with which the latching element engages, wherein the latching element can slide along in the grooves. The grooves can be arranged in the wall of the advancing element which may be cylindrical or sleeve-shaped. In the area in which the latching element is situated, a stopper element can be formed by the advancing element. The latching element can slide along the stopper element, wherein the latching element encompasses the stopper element via its latching portions. The latching element can be U-shaped and can encompass the stopper element via its limbs or arms, wherein the limbs can slide in the guiding grooves of the advancing element. The limbs can taper in the direction pointing towards the blocking element, whereby the blocking element is made easier to grip behind when the blocking element is latched into the blocking groove of the mechanism holder. The portion of the latching element via which the latching element is accommodated in the grooves can exhibit a greater distance from the longitudinal axis of the device than the latching portion. The surface of the latching portion which points radially away from the longitudinal axis, e.g. the surface which holds the at least one blocking element in the blocking groove, can be approximately parallel to the longitudinal axis. The latching portion and the portion via which the latching element is accommodated in the grooves can be connected by the tapered portion.

In some preferred embodiments, the latching element can be driven by an elasticity means or structure, e.g. a spring, which is supported on an element which is fixed to the advancing element and on the latching element. An appropriate spring can be, e.g., a helical spring, a combined helical and spiral spring, or a leaf spring. Equally, the element which is fixed to the advancing element can form the spring. The spring can be pressure-biased, whereby a force acts on the latching element in a direction, e.g. in the distal direction, and attempts to shift the latching portion in front of the blocking element. The latching element is in axial contact with the advancing element, e.g. with a stopper element or the blocking element, when the movement of the blocking element out of the engagement with the mechanism holder is blocked by the latching element. The tapered portion can abut the blocking element, e.g. a gear surface formed on it. Alternatively, the portion connecting the limbs of the latching portion can come into contact with the stopper element of the advancing element.

In some preferred embodiments, a driven member is provided which can be moved along the longitudinal axis relative to the advancing element when the blocking element engages with the mechanism holder. The driven member can be sleeve-shaped and can slide along the advancing element within the advancing element. An annular gap can be situated between the inner circumferential surface of the advancing element and the outer circumferential surface of the driven member. A proximal end of the driven member can comprise a collar which projects from the longitudinal axis in an approximately radial direction and can be in axial contact with the blocking element of the advancing element, such that a movement of the driven member in the delivery direction is blocked or prevented when the blocking element is latched out of the blocking groove. The collar projects radially from the outer circumferential surface of the driven member to an extent which is smaller than the radial width of the annular groove between the driven member and the advancing element.

In some preferred embodiments, the blocking element prevents the movement of at least one of the driven member and the latching element relative to each other or to the advancing element, along the longitudinal axis, when the blocking element is not in the engagement with the mechanism holder, wherein a force can act on the driven member, by which the driven member can be driven. The force can be generated by an elasticity element, e.g. a spring, which can be pressure-biased. A helical spring is suitable for this. The elasticity element is accommodated within the sleeve-shaped driven member. In some preferred embodiments, the elasticity constant or spring constant of the elasticity means (e.g., a suitable spring) which can drive the advancing element is greater than the elasticity constant or spring constant of the elasticity means (e.g., a suitable spring) which can drive the driven member, wherein the use of a latching element is advantageous if the device is re-activated after an injection.

In some embodiments, the elasticity element for driving the driven member can be supported on the driven member and on the advancing element, e.g. on the stopper element.

In some embodiments, at least one of the driven member and the latching element can be pressed into an axial contact with the blocking element by being respectively spring-driven or urged. In some preferred embodiments, the driven member is in direct contact with the blocking element and the latching element is in direct contact with the driven member, such that the driven member can be situated between the blocking element and the latching element when the blocking element is not in an engagement with the mechanism holder. The blocking element can comprise a gear surface which is inclined towards the longitudinal axis of the injection device, such that a longitudinal movement, e.g. in the distal direction, by at least one of the driven member and the latching element can move the at least one blocking element radially into the engagement with the latching groove of the mechanism holder, when the blocking element is at approximately the same axial height as the latching groove.

In some preferred embodiments, the gear surface forms the axial stopper for the driven member and/or latching element. The blocking element can engage with the blocking groove against a spring force which acts on the blocking element, counter to its engaging movement direction. The collar of the driven member presses the blocking element into the engagement with the mechanism holder, wherein the following latching element is shifted in front of the blocking element and prevents the blocking element from being released from the engagement with the mechanism holder. The collar of the driven member can comprise a surface which is inclined towards the longitudinal axis, e.g. to the same degree as the gear surface of the blocking element is inclined towards the longitudinal axis, such that the gear surface and the inclined surface of the collar lie approximately flat against each other when sliding off. The at least one latching portion of the latching element can form the stopper for co-operating with the collar or the blocking element.

In some embodiments, the device can also comprise a switching element which can be moved along the longitudinal axis from an initial position into a triggering position via a coupling position. From when the switching element is in the initial position to when the switching element is in the coupling position, a blocking member which is arranged, for example elastically, on the mechanism holder is held by the switching element in an engagement with the advancing element, e.g. in a respective latching groove provided for the blocking member. The latching groove can be annular, wherein the at least one blocking member engages with the common annular latching groove. When the switching element is in the triggering position, the switching element releases—for example, radially outwardly—the blocking member which engages with the advancing element, such that the blocking member is moved out of the engagement with the latching groove, whereby the advancing element is moved in the injecting direction of the needle, for example by a biased spring. The advancing element slaves or moves the product container in the injecting direction, e.g. via a cage which can be longitudinally shifted, such that the needle is injected into a tissue.

In some embodiments, once an injection sequence has been completely performed, the at least one blocking element of the advancing element can latch into the mechanism holder as described, whereby the advancing element is axially blocked, the driven member is released for a delivery movement, and/or the latching element is shifted in front of the at least one blocking element of the advancing element. The delivery movement of the driven member is transmitted onto a piston in the product container via a piston rod, whereby the liquid—for example, a viscous liquid—product is delivered through the needle into a tissue.

In some embodiments, the present invention relates to a method for preparing an injection device for an injection and delivery sequence, wherein it is assumed that the device is in post product delivery state, i.e., it has been performed a product delivery. The driven member is moved counter to the direction for the product delivery, wherein it is moved relative to the advancing element until the driven member firstly slaves or moves the latching element, whereby the blocking element is released for movement, and then slaves or moves the advancing element, wherein the blocking element which is arranged on the advancing element is moved out of the engagement with the mechanism holder and into an engagement with the driven member. The blocking element is moved into the engagement with the driven member such that the driven member can only be retracted in the delivery direction by a part of the path traveled counter to the delivery direction when sliding back. The advancing element is retracted until the blocking element of the mechanism holder is at approximately the same height, in the longitudinal direction, as the blocking groove of the advancing element, such that the blocking element is moved into the latching groove, which blocks or prevents the movement of the advancing element in the injecting direction. By engaging the blocking element of the mechanism holder, the switching element can be retracted from the triggering position into the initial position, via the coupling position. The device is then ready for a new injection.

In some embodiments, the present invention also relates to a method for controlling an injection sequence, wherein the advancing element is moved along the longitudinal axis until at least one blocking element which is coupled to the advancing element engages with a mechanism holder, such that a movement of the advancing element relative to the mechanism holder is blocked or prevented. At approximately the same time as the at least one blocking element engages with the mechanism holder, the latching element is released. The latching element is shifted along the longitudinal axis into a position in front of the at least one blocking element, such that a movement of the at least one blocking element out of the engagement with the mechanism holder is blocked.

### BRIEF DESCRIPTION OF THE DRAWINGS

FIG. 1 is a sectional representation along a longitudinal axis of one embodiment of an injection device in accordance with the present invention;

FIG. 2 is a sectional representation along a longitudinal axis of an embodiment of an injection device in accordance with the present invention;

FIG. 3 is a sectional representation along the longitudinal axis, perpendicular to the sectional representation of FIG. 2;

FIG. 4 is a perspective view of an embodiment of a switching element and a mechanism holder;

FIG. 5 is a sectional view of a part of an embodiment of an injection device in L accordance with the present invention in an initial state;

FIG. 6 is a sectional view of the part of the injection device of FIG. 5, after a product delivery;

FIG. 7 is a sectional view of an embodiment of a switching element and a triggering element, in an initial position;

FIG. 8 is a sectional view of the switching element and the triggering element of FIG. 7, in a coupling position;

FIG. 9 is a sectional view of the switching element and the triggering element of FIGS. 7 and 8, in a release position; and

FIG. 10 is an exploded representation, including embodiments of an advancing element, a latching element and an elasticity means.

### DETAILED DESCRIPTION

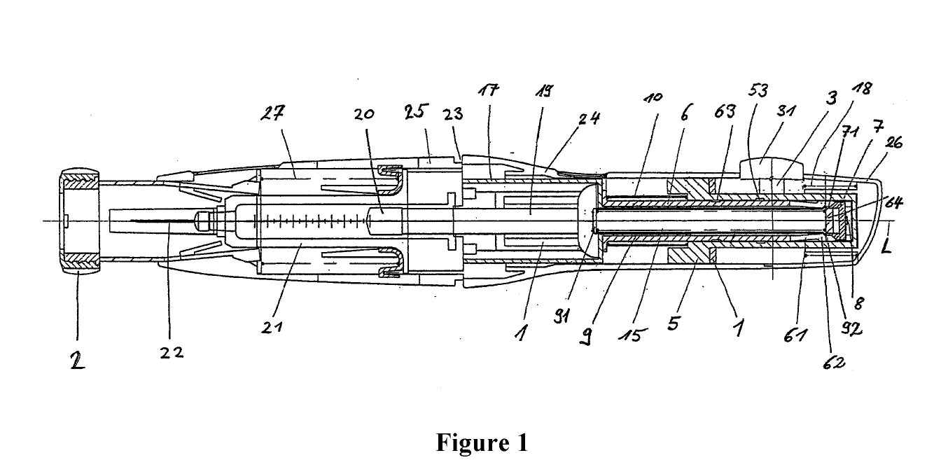

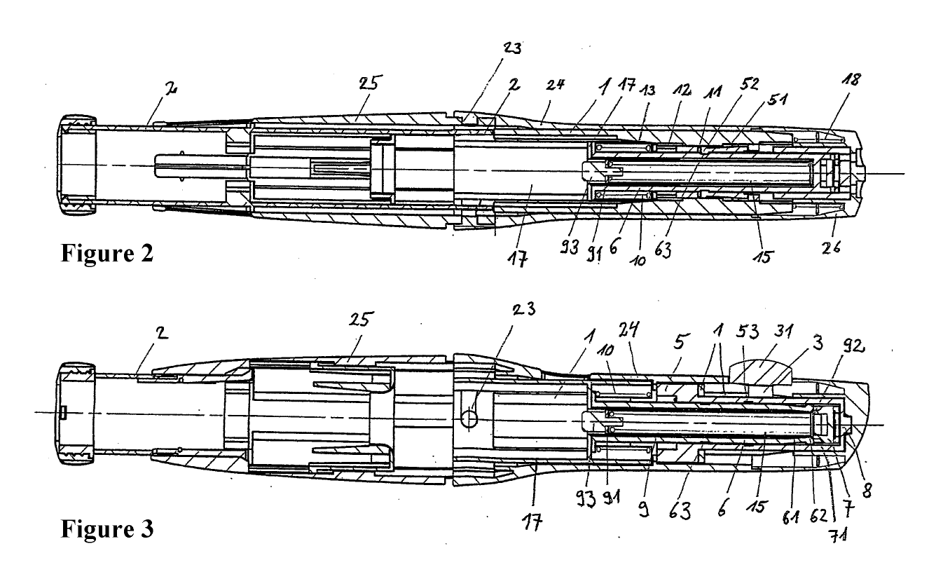

FIG. 1 shows an injection device in the form of an auto-injector. The injection device comprises a mechanism including a mechanical sequence controller, using which a product container 21 contained in the injection device is firstly shifted in the distal direction during an injection sequence, such that the needle 22 fixed to the product container 21 is injected into a body tissue of a patient, wherein after the injection sequence, the piston 20 in the product container 21 is moved in the distal or forward or injection direction during a delivery sequence, such that the product contained in the container 21 is delivered through the needle 22 into the body or body tissue of a patient. Unless specified otherwise, the description with respect to FIG. 1 also relates to FIGS. 2 and 3, in which a similar injection device is shown, without a product container 21.

The injection device comprises a distal housing part 25 and a proximal housing part 24 which are connected by a detachable connection, e.g. a bayonet lock 23. A mechanism holder 5 is accommodated in the proximal housing part 24 and connected, rotationally and axially fixed, to the proximal housing part 24. To axially fix the mechanism holder 5, it is axially enclosed via its facing sides between a protrusion of the housing 24 which protrudes radially inwardly and a cap 26 which is proximally fitted onto the housing 24. The mechanism holder 5 encompasses an advancing element 6 which can be shifted relative to the mechanism holder 5 along the longitudinal axis L of the injection device. The outer circumferential surface of the sleeve-shaped advancing element 6 can slide along the inner circumferential surface of the approximately sleeve-shaped mechanism holder 5. A spring 10, e.g. a helical spring, is arranged between a distal or forward facing side of the mechanism holder 5 and a proximal or rearward facing side of an annular flange arranged on the distal end of the advancing element 6, and when pressure-biased, the spring charges or urges the advancing element 6 with an advancing force in the distal direction relative to the mechanism holder 5.

The distal facing surface of the annular flange which is distally attached to the advancing element 6 can act, in a direction along the longitudinal axis L, on a cage 17 which can be shifted in the housing along the longitudinal axis L. The cage 17 is coupled to the product container 21 such that when the cage 17 moves in the distal direction, the product container 21 is likewise moved in the distal direction, i.e. in the injecting direction of the needle 22. The movement of the cage 17 in the distal direction cannot yet cause a movement of the piston 20 within the product container 21. The product container 21 and the cage 17 are pressed in the proximal direction of the injection device by a pressure-biased spring 27. The force of the spring 27 with which the cage 17 is pressed in the proximal direction is smaller than the force of the spring 10 with which the advancing element 6 and the cage 17 can be moved in the distal direction when the advancing element 6 is released for moving along the longitudinal axis L, such that the product container 21 can be moved together with the needle 22 in the distal direction to inject the needle 22 into the body tissue.

A sleeve-shaped driven member 9 is arranged within the advancing element 6 and can be moved relative to the advancing element 6 along the longitudinal axis L for a delivery sequence, when it is released for axial movement. A collar 92 which protrudes radially outwardly is situated on the proximal end of the driven member 9. An annular groove is situated between the driven member 9 and the advancing element 6 and has a groove width which is greater than the extent to which the collar 92 projects radially from the driven member 9. To form the annular groove and to mount or carry the driven member 9, a sleeve-shaped portion of the cage 17 engages from the distal side with the inner diameter of the advancing element 9, as can best be seen in FIG. 5.

The distal end of the driven member 9 comprises a facing surface 91 which acts as a stopper surface on which a spring 15, e.g. a helical spring, can be supported. The other end of the spring 15, which can be pressure-biased, is supported on a distal surface of a stopper element 64 which is formed on a proximal area of the advancing element 6. The spring 15, which exhibits a greater spring constant than the spring 10, can drive the released driven member 9 in the distal direction along the longitudinal axis L. The distal facing side of the driven member 9 or a contact element 93 fixed to it, as shown in FIGS. 2 and 3, come into contact with a piston rod 19 or a domed element arranged on the proximal end of the piston rod 19, whereby the piston 20, which is distally arranged on the piston rod 19, is moved in the product container, whereby the product is delivered through the needle 22 when the driven member 9 is moved in the distal direction.

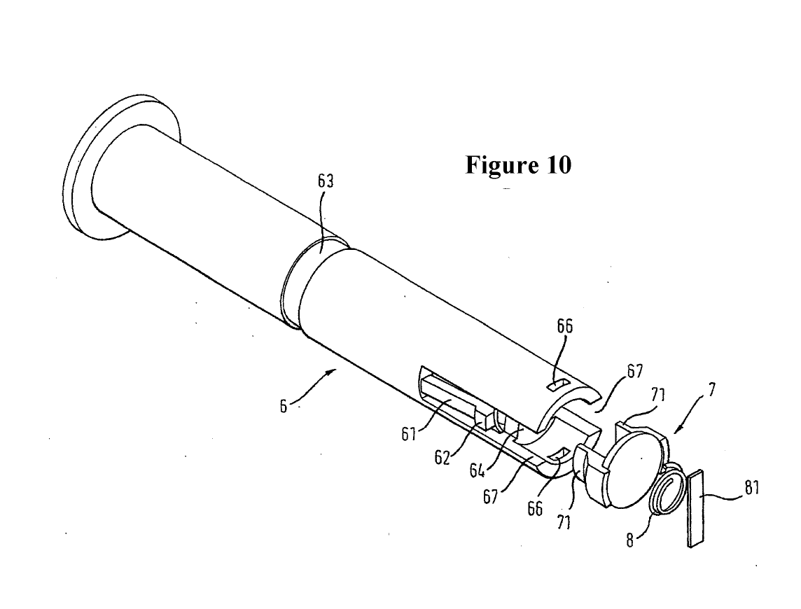

As can best be seen in FIG. 5, a latching element 7 which can be moved relative to the advancing element 6 along the longitudinal axis L is arranged on the proximal end of the advancing element 6. As can be seen in FIG. 10, the latching element 7 is forked and encompasses the longitudinal axis L via two latching portions 71. The latching portions 71 are each guided in a groove-shaped guide 67 (see FIG. 10) formed by the advancing element 6. The latching element 7 is also guided by a stopper element 64 which is formed by the advancing element 6 and accommodated between the latching portions 71. As shown, for example, in FIG. 5, a distance d can exist between the proximal facing side of the stopper element 64 and a distal facing side of the latching element 7, over which the latching element 7 can be moved in the distal direction. As can be seen in FIG. 10, the latching element 7 is charged or urged with a force in the distal direction by a spring 8. The spring 8 is supported on the proximal end of the latching element 7 and on an element which is arranged proximally with respect to it and fixed to the advancing element, i.e. on the holding stay 81. The holding stay 81 can be inserted into the advancing element 6, transverse to the longitudinal axis L, through openings which form a holding stay receptacle 66, and fixed to the advancing element 6 to serve as an abutment for the spring 8. The spring 8 can be a helical spring, a combined helical and spiral spring, or a leaf spring. In some embodiments, the holding stay 81 can be formed from a spring material and can exhibit a shape which can charge the latching element 7 directly with a spring force, such that the spring 8 can be omitted, since the latching element 7 assumes the function of the spring 8.

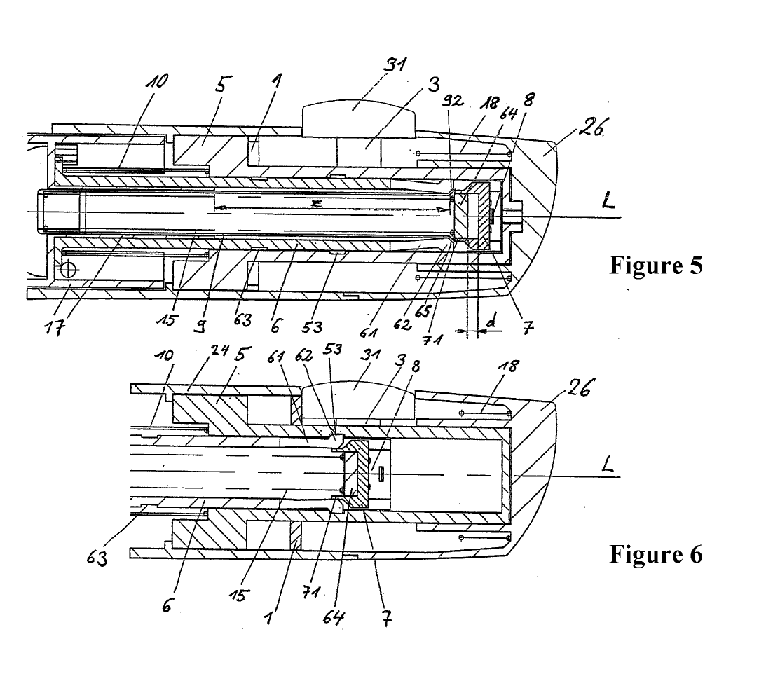

In the position shown in FIG. 5, a movement of the latching element 7 in the distal direction is prevented by the latching element 7 being blocked via its latching portions 71, each by a gear surface 65 of a blocking element 62. The cam-shaped or tapered blocking element 62 is arranged on an arm 61 and may be integrally connected, elastically, to the advancing element 6. The gear surface 65 simultaneously serves to block the longitudinal movement of the driven member 9, in the position shown in FIG. 5. To this end, the collar 92 of the driven member 9 abuts the gear surface 65. The latching element 7 can either abut the gear surface 65 directly or abut a proximal portion of the collar 92. If the released advancing element 6 is moved in the distal direction, the blocking element 62 arrives at approximately the same axial height as a latching groove 53, after a certain path length which approximately corresponds to the path over which the product container 21 is shifted for injecting. The latching groove 53 is formed by the advancing element 6. The latching groove 53 can be a recess or an annularly circumferential recess for each of the blocking elements 62. As soon as the latching groove 53 and the blocking element 62 are at the same axial height, as shown in FIG. 6, the spring force of the arms 61, the spring force of the spring 8, the spring force of the spring 15 in conjunction with the collar 92, and/or the latching element 7 sliding off on the gear surface 65 of the blocking element 62 causes a movement radially outwardly, into the latching groove 53. This starts the delivery sequence.

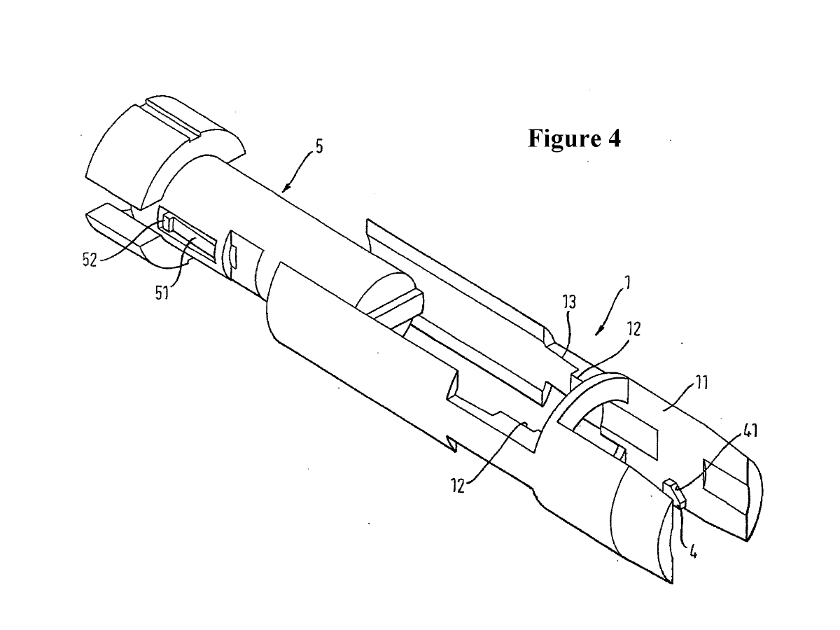

The collar 92 is then no longer blocked by the gear surface 65, whereby the spring 15 drives the driven member 9 in the distal direction and thus drives the piston 20 for a product delivery. The latching element 7 is simultaneously released for axial movement, such that the spring 8 shifts the latching element 7 over the path d (FIG. 5), whereby the latching portions 71 are shifted in front of the blocking elements 62. The blocking element(s) 62 is prevented from moving out of the latching groove 53, since the latching portion 71 blocks the movement which the blocking element 62 would perform if it moved out of the latching groove 53. The movement of the driven member 9 can be limited to a path z (FIG. 4), wherein the sleeve-shaped portion of the cage 17 forms the end stopper, or limited to a path which the piston 20 can travel in the product container 21.

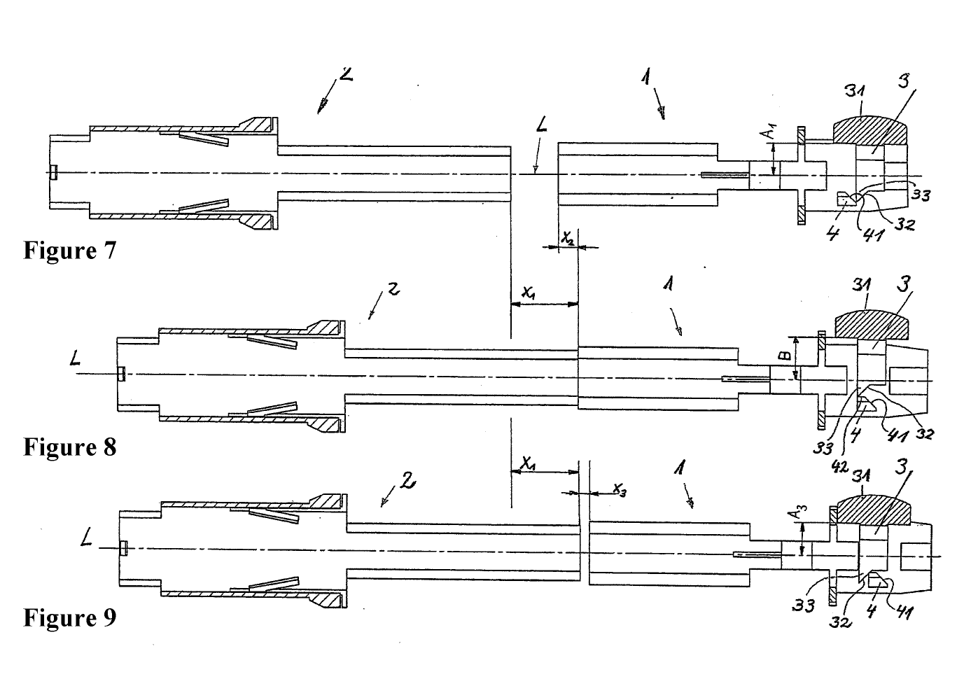

In some preferred embodiments, the delivery movement of the driven member 9 can only be performed once the needle 22 has completely performed its injection movement. In some embodiments, the injection sequence can only be started once a transmission member 2, formed as a needle cover, has been pressed sufficiently firmly onto the body tissue at the point of injection. When the device is pressed onto the body tissue, the transmission member 2 is shifted relative to the distal housing part 25. The proximal housing part 24 mounts a switching element 1 such that it can be moved along the longitudinal axis L. As shown in FIGS. 7 to 9, the switching element 1 is slaved or moved by the movement of the transmission member 2 in the proximal direction, wherein a proximal facing surface of the transmission member 2 and a distal facing surface of the switching element 1 come into contact. The transmission member 2 is moved in the proximal direction, counter to the force of a spring. The switching element 1 is pressed in the distal direction by a spring 18. The switching element 1 can be moved in the proximal direction, against the force of the spring 18.

The switching element 1 comprises an annular portion which co-operates with the mechanism holder 5 as an axial stopper for a movement of the switching element 1 in the distal direction, when the switching element 1 is in its initial position. The switching element 1 comprises a guiding surface element 4 which comprises a guiding surface 41 which is inclined towards the longitudinal axis L. As shown in FIG. 4, the switching element 1 is forked. A guiding surface element 4 is situated on each of the two fork elements. A triggering element 3, which is likewise forked, comprises a cam 33 on each of its fork elements, which can co-operate with the respective guiding surface element 4. The triggering element 3 can be moved transversely—as shown here—perpendicularly, to the longitudinal axis L. The cams 33 and the guiding surface elements 4 are each arranged at a distance from a plane which is spanned by the longitudinal axis L and the movement direction of the triggering element 3. The fork elements of the switching element 1 and the fork elements of the triggering element 3 encompass the longitudinal axis L.

FIG. 7 shows an initial position of the switching element 1. The triggering element 3 is pressed against the guiding surface 41 of the guiding surface element 4 via the cam 33 by an elasticity element, e.g. a spring (not shown), wherein an actuation element 31 connected to the triggering element 3 assumes a position below or flush with the height of the housing, as shown in FIG. 6, wherein the position shown in FIG. 6 is an example. When the switching element 1 is in the position shown in FIG. 7, the actuation element 31 cannot or not immediately be actuated, e.g. by a finger of the user. Actuating the actuation element 31 when the switching element 1 is in the initial position shown in FIG. 7 or in an intermediate position between the initial position and the coupling position does not lead to the sequence controller for the injection being triggered.

When the user of the device presses the transmission member 2 onto the point of injection, it is shifted over or along the path x₁, whereby the switching element 1 is slaved or moved over or along the path x₂. During this movement, the guiding surface 41 which is inclined towards the longitudinal axis L slides along the cam 33, whereby the triggering element 3 is firstly shifted transversely to the longitudinal axis L, such that the actuation element 31 rises above the outer surface of the housing 25, whereby the user can access the actuation element 31 or derive an optical indication that the device is ready for triggering the sequence controller, such that the user can subsequently actuate the triggering element 3. When the switching element 1 is moved further in the proximal direction, a guiding surface 42 travels along the cam 33, wherein the guiding surface 42 runs approximately parallel to the longitudinal axis L, as shown in this example, such that when the guiding surface 42 moves along the cam 33, the triggering element 3 remains stationary. Once the switching element has reached the coupling position shown in FIG. 8, the triggering element 3 or actuation element 31, respectively, can be actuated to effectively trigger the sequence controller.

When, as shown in FIG. 9, the actuation element 31 is pressed transversely to the longitudinal axis L, the guiding surface 32 of the triggering element 3 then slides off on the guiding surface element 4, whereby the switching element 1 is moved over a path X₃ in the proximal direction, such that it assumes a release position. In the release position, the sequence controller of the injection device is started.

As shown in FIGS. 2 and 4, the mechanism holder 5 comprises a blocking member 52 which is elastically arranged via an arm 51 and engages with a latching groove 63 situated on the outer circumference of the advancing element 6. As long as the blocking member 52 engages with the latching groove 63, a movement of the advancing element 6 relative to the mechanism holder 5 is blocked or prevented. When the switching element 1 is in the initial position and in the coupling position, a holding stage 11 formed by the switching element 1 is at the same axial height as the blocking member 52, such that the blocking member 52 is held in engagement with the advancing element 6 by the holding stage 11. When the switching element is moved from the coupling position into the release position, a release stage 12 formed by the switching element 1 is moved to the same axial height as the blocking member 52, whereby the blocking member 52 is pressed out of engagement with the blocking groove 63 due to the elastic arm 51 and/or due to the gear action of the advancing element 6, which is charged with an axial force of the spring 10, and the geometry of the blocking member 52. The distance between the holding stage 11 and the longitudinal axis L is smaller than the distance between the release stage 12 and the longitudinal axis L. Mounting ramps 13 are arranged on the switching element 1 in the extension of the release stage 12 in the distal direction, on which the blocking members 52 are guided onto the holding stages 12 when the switching element 1 is mounted to the mechanism holder 5.

When the blocking members 52 are extended, the advancing element 6 is pressed in the distal direction by the spring 10 until the blocking elements 62 engage with the blocking groove 53, wherein the needle 22 is injected. Once the blocking elements 62 have engaged with the latching groove 53, the delivery sequence described above follows.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising:
a mechanism holder;
an advancing element that can be displaced along a longitudinal axis in relation to the mechanism holder;
a spring for driving the advancing element;
a blocking element associated with the advancing element and engageable in the mechanism holder to prevent a longitudinal movement of the advancing element in relation to the mechanism holder; and
a lock element that can be displaced along the longitudinal axis in relation to the advancing element, wherein in one position the lock element prevents the blocking element from disengaging from the mechanism holder.

2. An injection device comprising:
a) a mechanism holder;
b) an advancing element moveable along a longitudinal axis relative to the mechanism holder;
c) elasticity means for driving the advancing element;
d) at least one blocking element coupled to the advancing element and engageable with the mechanism holder to prevent a longitudinal movement of the advancing element relative to the mechanism holder; and
e) a latching element moveable along the longitudinal axis relative to the advancing element, wherein in one position relative to the at least one blocking element the latching element prevents a movement of the at least one blocking element out of the engagement with the mechanism holder.

3. The injection device according to claim 2, further comprising a driven member moveable along the longitudinal axis relative to the advancing element when the blocking element engages with the mechanism holder.

4. The injection device according to claim 3, wherein the blocking element prevents the movement of at least one of the driven member and the latching element relative to each other, along the longitudinal axis, when the blocking element is out of the engagement with the mechanism holder.

5. The injection device according to claim 3, wherein at least one of the driven member and the latching element can be in an axial contact with the blocking element.

6. The injection device according to claim 5, further comprising an axial stopper formed by a gear surface inclined towards the longitudinal axis, such that a longitudinal movement by at least one of the driven member and the latching element can move the at least one blocking element radially into the engagement with a latching groove of the mechanism holder, when the blocking element is at approximately the same axial height as the latching groove.

7. The injection device according to claim 3, wherein the driven member comprises a collar comprising a surface which is inclined toward the longitudinal axis, and the latching element comprises at least one latching portion, via which surface and portion the collar and latching element are in an axial contact with the extended blocking element.

8. The injection device according to claim 2, wherein the blocking element is situated between a groove base of a latching groove of the mechanism holder and a latching portion of the latching element when the blocking element is in an engagement with the mechanism holder.

9. The injection device according to claim 2, wherein the latching element is in axial contact with a stopper associated with the advancing element or with the blocking element when the movement of the blocking element out of the engagement with the mechanism holder is blocked by the latching element.

10. The injection device according to claim 2, wherein the advancing element comprises at least one guide along which the latching element can slide.

11. The injection device according to claim 3, wherein the driven member is driven by a spring supported on the advancing element and on the driven member.

12. The injection device according to claim 3, wherein the driven member is driven by an elasticity element accommodated within the driven member.

13. The injection device according to claim 12, wherein the advancing element is driven by a spring supported on the mechanism holder and on the advancing element.

14. The injection device according to claim 13, wherein the spring constant of the spring which drives the advancing element is greater than the spring constant of the spring which drives the driven member.

15. The injection device according to claim 2, wherein the latching element is driven by a spring supported on an element fixed to the advancing element and on the latching element.

16. The injection device according to claim 2, wherein actuating a triggering element releases the advancing element for an injection movement.

17. The injection device according to claim 6, wherein when the advancing element is shifted in the distal direction the blocking element can latch into the blocking groove, wherein when the blocking element is latched in, a relative movement between the advancing element and the mechanism holder is blocked and the driven member is released for a delivery movement.

18. A method for preparing an automatic injection device for an injection and product delivery sequence, the method comprising the steps of moving a driven member which is moveable in a delivery direction counter to the delivery direction and relative to an advancing element until the driven member firstly slaves a latching element whereby a blocking element is released for movement and then slaves the advancing element whereby the blocking element is moved into engagement with the driven member such that a complete delivery movement of the driven member in the delivery direction is prevented.

19. A method for controlling an injection sequence of an injection device, the method comprising the steps of:
a) moving an advancing element along a longitudinal axis until at least one blocking element coupled to the advancing element engages with a mechanism holder, thereby preventing a movement of the advancing element relative to the mechanism holder;
b) engaging the at least one blocking element with the mechanism holder to release a latching element; and
c) shifting the latching element along the longitudinal axis into a position relative to the at least one blocking element thereby preventing a movement of the at least one blocking element out of the engagement with the mechanism holder.
